(19) United States
(12) Patent Application Publication
(10) Pub. No.: US 2020/0038562 A1
(43) Pub. Date: Feb. 6, 2020

(54) Novel Implantable Devices And Related Methods And Materials

(71) Applicant: Bruce H. Levin, Oceanside, NY (US)

(72) Inventor: Bruce H. Levin, Oceanside, NY (US)

(21) Appl. No.: 16/529,738

(22) Filed: Aug. 1, 2019

Related U.S. Application Data
(60) Provisional application No. 62/713,383, filed on Aug. 1, 2018.

(51) Int. Cl. A61L 31/14; A61L 31/00; A61B 90/00; A61D 99/00

(52) U.S. Cl. CPC A61L 31/14; A61L 31/005; A61B 34/73; A61D 99/00; A61B 90/00

(57) ABSTRACT

The present invention provides implantable devices and related methods configured to stimulate, or otherwise modulate neural or other biological tissues or structures by implanting substance carrying devices and components such as micro-chips to promote, inhibit, alter, and otherwise affect and modulate the target structure's functionality, growth, expression, appearance and other features.

### RELATED APPLICATIONS

This application claims priority to U.S. Provisional Patent Application Ser. No. 62/713383 filed Aug. 1, 2018 titled the same and incorporation herein as if set out in full.

### BACKGROUND OF THE INVENTION

Microstimulation of neural tissue has been known for controlling neuronal responses. The present disclosure describes devices and related methodologies for modulating biological structures such as native or biologically engineered nerve tissue, culture, cultured tissue to be grated other tissue, tissue components, organs, muscles as well as non-biological structures to achieve desired results.

### SUMMARY OF THE INVENTION

Devices and methodologies of the invention are operable by guiding devices or component to targeted area. The devices or component may be conductive or non-conductive, liquid, fluid, gel, sol, gel-sol, malleable solid, foam, putty, or solidifying substance. May be pH, ion, temperature, light, or chemically dependent for state, volume, size, shape, conductivity, or transmissibility. The devices or component may contain one or more preformed electrode, diode, lead, circuit, integrated circuit, microcircuit, microelectronics, integrated circuits, carbon fiber nanotubules micro wires or metallic foci or magnetic foci, MEMS, wire, micro wire, array, deployable array, carbon fiber, carbon, carbon monolayer silicone, metal, rare metal, sensor or RFID device or related device or structure, circuit, nano-tubule, or other microcircuit, machine, power generator, power source, microrotor, magnet, battery, lead, electrode, sensor, generator or conduit for energy, including electricity, electromagnetics, magnetics, ultrasound, radio wave, radiation, light, laser or maser. The device may contain tissue, organ or organ component, chemotherapeutic agent, or radiotherapeutic agent or reservoir.

A substance may be used to contain the device or devices which maybe artificial or biologic, may be a hyaluronic acid, Restylane, hyaluronic acid variant, collagen or collagen like substance, cartilaginous substance, or it may be a form of neural, smooth or skeletal muscle, connective tissue, cartilaginous, vascular, endo or epithelial tissue. It may be genetically engineered or otherwise altered to enhance or otherwise effect certain physical, chemical, structural, conductive or other properties and/or to maintain or optimize homeostatic, metabolic, status or durability. It may be homogenous or nonhomogeneous.

The device may be directed into place by magnet or magnetism, electric field, electricity, heat, sound, ultra or infrasound, vibration, buoyancy, or light, concentration gradient, or by microbe. The device or devices maybe placed or introduced directly via needle, catheter or cannula, or shot to the desired location via pressure, mechanical, magnetic or electrical modality. The device or devices may be filled with, or be placed within a deployable, sealable or nonsealable, package or container in the form of a collapsible or non-collapsible structure, bag, balloon, polyhedron, sphere, ovoid, sealed stent-like container or other expansible container which can be made to increase in size.

In one exemplified application, the devices and methods are used in neuroaugmentation. In another embodiment, the devices and methods are used in cultures, native, or biologically engineered nerve tissue, or tissue components can be used as wire, conduit or to stimulate the affected neural structure being treated. Similarly, atrophied, scarred, damaged, contracted or otherwise defective muscle or other tissue can be replaced by cultured tissue and be stimulated to function or be graft connected to native neural or other structures via a cultured bridge of neural tissue, vascular tissue and the like.

In yet another embodiment, the methods and devices are used to treat diabetes by placing the stimulator on or in proximity to pancreatic neuropathways, within or in proximity to implanted units of beta cells to modulate insulin production, or as a micro vile or microdispensory device to regulate the secretion of insulin. In those with inadequate beta cell activity, beta cells native, autologous, non-autologous, cultured may be placed within tissue directly, following balloon or other ablation, directly therein or in a container permeable to insulin but nonpermeable to immune system components or beta cell antigens. This method is not to be limited to diabetes, but can be used in the treatment of Parkinson's disease, adrenal pathologies, the pituitary disorders, atrial natriuretic peptide disorders, cardiovascular regulatory sensor regulation or other diseases amendable to an implantable therapeutic.

### BRIEF DESCRIPTION OF THE DRAWINGS

FIG. 1 is an exemplary example of injection of Hyaluronic Acid containing devices or components, for example micro-chips, around the Supraorbital and Supratrochlear nerves illustrating the present invention.

FIG. 2 is an exemplary example of injection of Hyaluronic Acid matrix containing devices or components, for example micro-chips, subcutaneously and above bone overlying a branch of the SPG illustrating the present invention.

FIG. 3 is an exemplary example of implantation of devices and/or components into autologous, harvested or tissue cultured cartilage or other biological matrix in a body locus illustrating the present invention.

FIG. 4 is an exemplary example of implantation of components into different stages of fertilized ovum or blastocyst to alter development, promote growth or to achieve other biological stimulations illustrating the present invention.

FIG. 5 is an exemplary example of intracellular implantation where intracellular waste product is collected or rendered harmless by an intraorganelle and/or intracellular device illustrating the present invention.

FIG. 6 is an exemplary example of microinjection of MEMS or micro devices into a cell organelle, the nucleus in this case, to chemically, mechanically, energetically or otherwise alter DNA, mRNA, RNA, nucleotide, or protein structure, reproduction, production or function illustrating the present invention.

FIG. 7 is an exemplary example of injections of one or more cells into a blastocyst, the cells containing devices or components which causing the cells to possess different properties than the cells in blastocyst, eventually leading to a desired different embryonic development illustrating the present invention.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

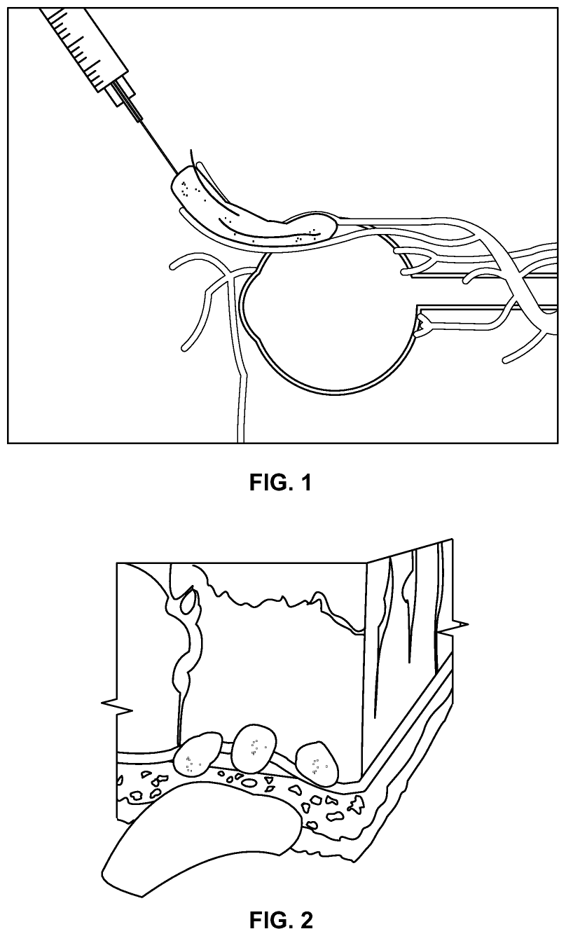

FIG. 1 is an exemplary example of injection of Hyaluronic Acid containing devices or components, for example micro-chips, around the Supraorbital and Supratrochlear nerves. The micro-chips may be substituted for MEMS, or any of the devices referenced in the present disclosure. Another embodiment could have the Hyaluronic Acid, or other substrates contain or include Micelles or liposomal pharmaceutical/nutraceutical/or other chemical agent instead of micro-chip.

FIG. 2 is an exemplary example of injection of Hyaluronic Acid matrix containing devices or components, for example micro-chips, subcutaneously and above bone overlying a branch of the SPG. The injection or placement can be below the overlying bone but not intravascular. Doppler or flow sensing needle can be used.

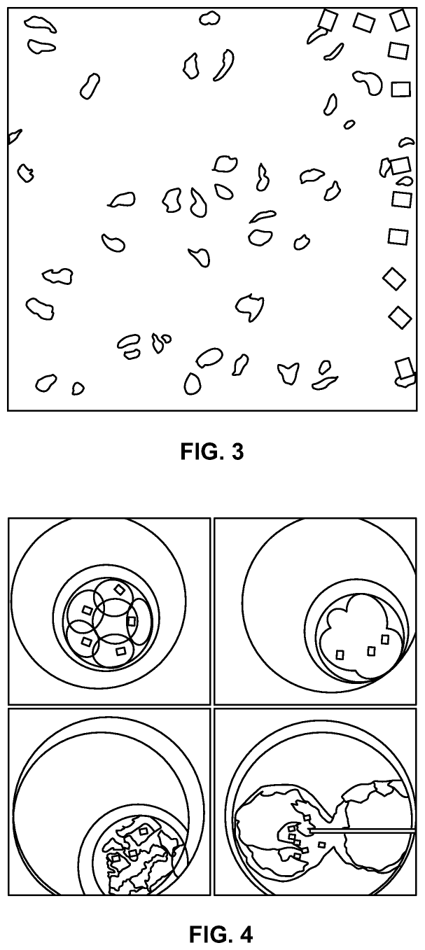

FIG. 3 is an exemplary example of implantation of devices and/or components into autologous, harvested or tissue cultured cartilage or other biological matrix in a body locus. The implant can be cut, shaped or otherwise formed to optimize configurationational function. It may be grown on a matrix scaffolding, or with 3-D printing. MRI, CT, C Ray, Ultrasound or other imaging can be used to optimize or guide size, shape and placement. Microchip or other device, or subunit can be located in the matrix in such a way as to optimize proximity and hence function of targeted anatomic structure.

FIG. 4 is an exemplary example of implantation of components into different stages of fertilized ovum or blastocyst to alter development, promote growth or to achieve other biological stimulations.

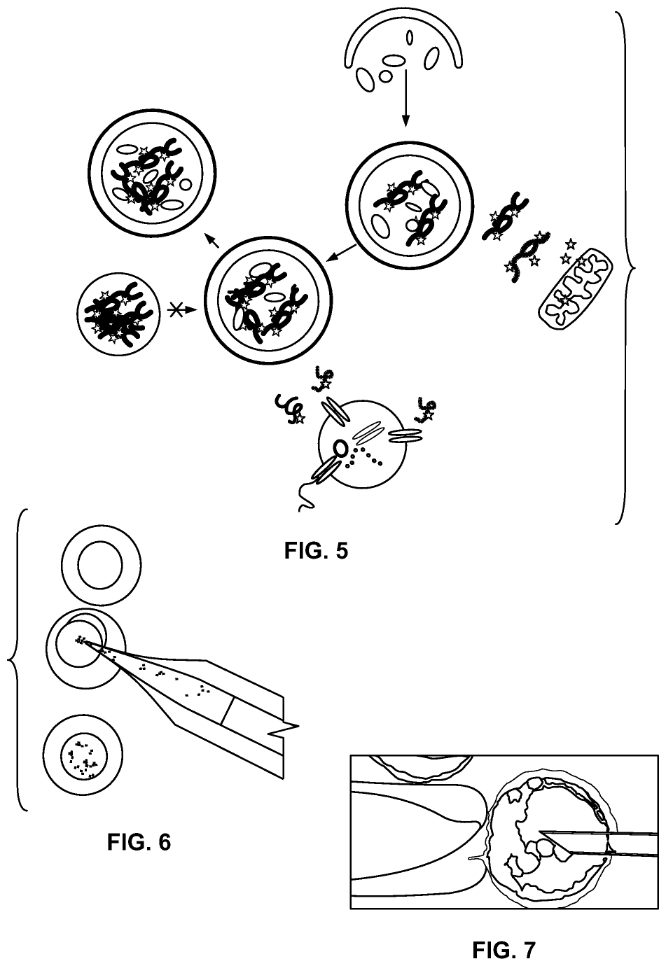

FIG. 5 is an exemplary example of intracellular implantation where intracellular waste product is collected or rendered harmless by an intraorganelle and/or intracellular device. In an exemplary example, devices or components are implanted into organelles such as lysosomes, autophagosomes, altering the effect of the waste product.

FIG. 6 is an exemplary example of microinjection of MEMS or micro devices into a cell organelle, the nucleus in this case, to chemically, mechanically, energetically or otherwise alter DNA, mRNA, RNA, nucleotide, or protein structure, reproduction, production or function. They also may be micro-introduced by microfilament individually or in groups by apical or radial detachment directly or following micro retraction of protective or enveloping micro sheath, or using a micro-Selinger mechanism. Similarly, the microinjection can also occur to Golgi Apparatus, endoplasmic reticuli etc.

FIG. 7 is an exemplary example of injections of one or more cells into a blastocyst, the cells containing devices or components which causing the cells to possess different properties than the cells in blastocyst, eventually leading to a desired different embryonic development. Devices such as nanomachines, MEMs, Stimulator or stimulators or the like can also be introduced into blastocysts or embryos. Similarly, the injection may also be to stem cells with stimulators or drug, hormone, nutrient or other materials.

The present disclosure can also be applied to pancreatic function, by microinjection to stimulate exocrine pancreas as well as endocrine pancreas to achieve nutritional homeostasis. The microinjection of devices and components can alter functions of releasing enzymes, (β-cells, α-cells, and δ-cells, be applied to treat diabetes. Similarly, the present disclosure can be applied to the CNS, such as the sympathetic and parasympathetic pancreatic enervation. The stimulation can be applied to one or more points at or along any location to alter endocrine or exocrine pancreatic function.

1. An implantable device implantable in humans, animals, plants, organs, tissues, tissue cultures, cell cultures, ova, gametes, eggs, hybrids, cells or organelles.

2. The method of claim 1, where the device consists of, or contains one or more of preformed electrode, diode, lead, circuit, integrated circuit, microcircuit, MEMS, wire, micro wire, array, deployable array, carbon fiber, carbon, carbon monolayer silicone, metal, rare metal, structure, circuit, nanotubule, or other microcircuit, machine, power generator, power source, microrotor, magnet, battery, lead, electrode, sensor, conduit for energy or electricity, electromagnetic array, generator of ultrasound, radiowave, radiation, light, laser, maser, or other energy, engineered cell or cells, biomanufactured organelle, modified or native organ or organ component, resevoir of chemotherapeutic agent, radiotherapeutic agent, sensor or RFID device or related device of MEMS, nanomachine, chip microchip, electrode, stimulator, sensor.

3. The method of claim 2, where the device itself contains artificial or biologic components, may include a collagen or collagen like substance, cartilaginous substance, a neural tissue or type tissue, a smooth or skeletal muscle tissue or type tissue, a connective tissue or type tissue, a cartilaginous tissue or type tissue, vascular tissue or type tissue, an endo or epithelial tissue or type tissue.

4. The method of claim 3, where the component is native or biologic tissue, cultured tissue, genetically engineered or otherwise altered tissue to enhance or otherwise effect certain physical, chemical, structural, conductive or other properties and/or maintain or optimize homeostatic, metabolic, status or function and durability.

5. The method of claim 2 wherein implantation is percutaneous, peri/trans/intravascular, peri/trans/intracavitary, peri/trans/intraluminal, luminal, dural, peridural, sub dural, intradural, intracranial, arachnoid, subarachnoid, meningeal, sub/intra/periventricular, neural, myofascial, adipose, skeletal or smooth muscle, or cardiac structure or tissue or intratissue.

6. The method of claim 5 where implantation is following or concurrent with volume clearing procedure via surgery, balloon plasty, ultrasound, heat, electrical, radiofrequency or other ablative or radiologic or gamma radiation techniques.

7. The method of claim 1, where one or more devices are embedded, suspended, placed, or physically associated with a substance or material consisting of conductive or non-conductive liquid, fluid, gel, sol, gelsol, malleable solid, foam, putty, or matrix, cell culture, autologous, homologous, cadaveric tissue, tissue culture, or solidifying substance.

8. The method of claim 7 where the substance or material is pH, ion, temperature, light, or chemically dependent for state, volume, size, shape, conductivity, or transmissibility.

9. Method of claim 7 where the substance or material is artificial or biologic, is hyaluronic acid, Restylane, hyaluronic acid variant, collagen or collagen like substance, cartilaginous substance, is a native or biologic tissue cultured or harvested, is a neural, smooth or skeletal muscle, connective tissue, cartilaginous, vascular, endo or epithelial tissue.

10. The method of claim 9, wherein structure of substance, material, and/or device is customized according to MRI, CT, X-ray, Ultrasound or other imaging or structure defining modality is utilized. This may include culture, scaffolding or 3D printing or other manufacturing modality.

11. The method of claim 7 where the material may be genetically engineered or otherwise altered to enhance or otherwise effect certain physical, chemical, structural, conductive or other properties and/or maintain or optimize homeostatic, metabolic, status or durability.

12. One or more stimulating devices or components directed into place by utilizing a magnet or magnetism, electric field, electricity, heat, sound, ultra or infrasound, vibration, buoyancy, or light, concentration gradient, or by microbe, or micromachine.

13. A device capable of stimulating or otherwise affecting neural or other biological tissues or structures, comprising
One or more of devices or components placed in a fluid or gel like substance or putty like substance.

14. The device of claim 13, wherein the one or more of devices or components are liquid or fluid.

15. The device of claim 13, wherein the one or more of devices or components are solid or solidified.

16. The device of claim 13, wherein the one or more of devices or components are in native form.

17. The device of claim 13, wherein the one or more of devices or components are arranged in a series configuration.

18. The device of claim 13, wherein the one or more of devices or components are in parallel configurations to each other.

19. The device of claim 13, wherein the one or more of devices or components are within one or more matrixes to fix their positions.

20. The device of claim 13, wherein the one or more of devices or components are magnetic.

21. The device of claim 13, wherein the one or more of devices or components areferrous.

22. The device of claim 13, wherein the one or more of devices or components are in magnetized fluid or liquid.

23. The device of claim 13, wherein the one or more of devices or components are fixed.

24. The device of claim 13, wherein the one or more of devices or components are detachable by electric, magnetic, spring, rotational, pressure or other modality.

25. The device of claim 13, wherein the structures or structures are one or more organelles.

26. A methodology utilizing a doppler or flow sensing needle or other introducer to guide proper placement and avoid vascular or other trauma when introducing medications, or other devices, performing biopsy or other procedures.

27. A method of stimulating or otherwise affecting neural or other biological tissues or structures, comprising
Placing one or more of devices or components in a fluid or gel like substance or putty like substance.

28. The method of claim 27, wherein the one or more of devices or components are liquid or fluid.

29. The method of claim 27, wherein the one or more of devices or components are solid or solidified.

30. The method of claim 27, wherein the one or more of devices or components are in native form.

31. The method of claim 27, wherein the one or more of devices or components are arranged in a series configuration.

32. The method of claim 27, wherein the one or more of devices or components are in parallel configurations to each other.

33. The method of claim 27, the one or more of devices or components are within one or more matrixes to fix their positions.

34. The method of claim 27, wherein the one or more of devices or components are guided by magnetic.

35. The method of claim 27, wherein the one or more of devices or components are guided to assemble.

36. The method of claim 27, wherein the one or more of devices or components are detachable by electric, magnetic, spring, rotational, pressure or other modality.

37. The device of claim 27, wherein the structure is one or more organelles.
